(19) United States
(12) Patent Application Publication
(10) Pub. No.: US 2011/0020799 A1
(43) Pub. Date: Jan. 27, 2011

(54) Screening method for damaged DNA repairing substance

(75) Inventors: Tomoo Ogi, Nagasaki (JP); Siripan Limsirichaikul, Nagasaki (JP); Yuka Nakazawa, Nagasaki (JP); Shunichi Yamashita, Nagasaki (JP)

Correspondence Address: WENDEROTH, LIND & PONACK, L.L.P., 1030 15th Street, N.W.,, Suite 400 East, Washington, DC, 20005-1503, US

(73) Assignee: NAGASAKI UNIVERSITY

(21) Appl. No.: 12/656,408

(22) Filed: Jan. 28, 2010

(30) Foreign Application Priority Data
Jul. 23, 2009 (JP) 172521/2009

(51) Int. Cl. C12Q 1/68

(52) U.S. Cl. 435/6

(57) ABSTRACT

Provided are a novel screening method for a substance that potentiates damaged DNA repair capability, based on a test with DNA repair as an index with improved sensitivity as a simplified version of the currently available unscheduled DNA synthesis (UDS) assay based on ³H-thymidine and BrdU or recovery of RNA synthesis (RRS) test, and the like. By measuring UDS activity quickly at high sensitivity using a method of nucleotide fluorescence detection with the use of a click chemistry reaction (e.g., detection of terminal alkyne-modified nucleoside by means of a reporter molecule containing an azide moiety), a substance capable of DNA repair can be selected.

### TECHNICAL FIELD

The present invention relates to a screening method for a substance or gene with damaged DNA repairing synthesis activity or inhibition of RNA synthesis due to DNA damage as an index. More specifically, the present invention relates to the detection of a damaged DNA repair site or inhibition of RNA m synthesis due to DNA damage by means of a click chemistry reaction, and to cytotoxicity tests for various substances, screening methods for a DNA damage suppressing effect, a DNA repair promoting effect, search for novel genes involved in DNA repair and the like, and a clinical diagnostic technique and is the like, with the damaged DNA repair site or inhibition of RNA synthesis due to DNA damage as an index.

### BACKGROUND ART

Organisms have developed their DNA repair mechanisms in order to protect and maintain genetic information against a wide variety of DNA damages induced by various factors, for example, ultraviolet rays and the like. These are mechanisms wherein damaged DNA is sensed, and the DNA is repaired by the activities of a series of enzymes having repairing functions to suppress the influence on the living organism. Nucleotide excision repair (NER) is one of the most ubiquitous DNA repair systems, corresponding mainly to photo-induced DNA damage by ultraviolet rays, and addition type DNA damage due to exposure to various chemical carcinogens.

Any abnormality and/or deficiency in a gene involved in DNA repair makes the repair of damaged DNA incomplete, resulting in the onset of various diseases, including cancers. Diseases caused by such abnormalities in DNA repair genes, particularly by abnormalities in NER-related genes, include xeroderma pigmentosum (XP), Cockayne's syndrome (CS) and trichothiodystrophy (TTD).

The most commonly used assay for evaluating deficiencies in DNA repair mechanisms, including NER, requires a measurement of nucleotide incorporation level due to DNA repair activity. This is to detect trace amounts of DNA synthesis that does not depend on the cell cycle, known as unscheduled DNA synthesis (UDS) or repairing DNA synthesis; various methods for quantifying UDS have been established to date. In evaluating NER activity, it is common practice to irradiate cells with ultraviolet light of 254 nm wavelength to induce DNA damage, and the cells thus treated to induce DNA damage are cultured in the presence of either radioactive thymidine or a nucleoside analogue thereof to determine nucleotide incorporation levels. In non-NER DNA repair mechanisms as well, the activities of a wide variety of DNA repairs accompanied by DNA synthesis can be measured by performing different treatments to induce DNA damage according to the features of the repair pathway.

The currently most widely used method of quantifying UDS at research facilities where clinical diagnoses are performed of XP, which is a deficiency of NER, is based on the incorporation of radioactive ³H-thymidine. This is a technique wherein the radioactive thymidine incorporated in the process of DNA repair is converted to silver particles by autoradiography, and the particles are counted under a microscope, or a technique wherein the radioactive thymidine incorporated is insolubilized, and its particles are counted using a liquid scintillation counter to determine the UDS activity. Although autoradiography provides accurate measurements of UDS activity, the experimental process is complex, requires high skills, and takes a long time of 2 to 3 weeks; furthermore, a facility where radioisotopes are utilized is essential. The method using a liquid scintillation counter takes shorter operating times than autoradiography, but this method is a batch assay, so the accuracy of UDS quantitation decreases. Furthermore, the necessity for complete elimination of cell cycle DNA synthesis makes it necessary to use cells in non-dividing phases for the assay in combination with hydroxyurea (HU), an inhibitor of DNA replication, to eliminate cell cycle DNA synthesis, which occurs at very low but detectable levels. For these reasons, this technique is employed at only a very limited number of research facilities.

The method for detecting BrdU incorporation by means of anti-BrdU antibody using bromodeoxyuridine (BrdU) in place of ³H-thymidine is more convenient than the method using ³H-thymidine. As the situation stands, however, there is a detection sensitivity issue (UDS activity reductions of 50% or less are undetectable), so application of this method to methods of UDS assay and practical use as a diagnostic method for XP have not yet been realized. Although genetic tests by PCR amplification and base sequencing of already identified XP-causal gene loci, an antibody against XP protein and the like have been developed (JP-A-2006-296287), there are at least 11 XP-causal genes; genetic tests and/or antibodies for the products of all these genes are required in clinical diagnoses, so the problem of procedural complexity remains unsolved.

A diagnostic technique for NER repair deficiency used in combination with UDS assay is the recovery of RNA synthesis (RRS) test. This is to measure the activity of DNA repair in concomitance with RNA transcription (TCR) in NER only, and is utilized for diagnosing Cockayne's syndrome and other conditions involving TCR deficiency. If an abnormality is present in TCR only, the repair involving the global genome (GGR), which accounts for about 90% of total NER activity, is normal, so the UDS activity exhibits nearly normal values. However, because the repair of genes in vigorous action of RNA transcription is inefficient, RNA synthesis activity after DNA damage decreases considerably. In measuring RRS, radioactive uridine and the like are used; the extent of recovery of RNA synthesis after treatment to induce DNA damage is determined by a batch assay using a liquid scintillation counter.

Meanwhile, the click chemistry reaction, proposed by K. B. Sharpless et. al., makes it possible to bind two molecules via a carbon-hetero-atom bond through a nucleophilic addition ring opening reaction, condensation reaction, addition cyclization reaction or the like, and is expected to contribute to the creation of novel functional molecules. In particular, the addition cyclization reaction of an azide and alkyne compound is highly specific and offers advantages, including high percent yields of desired product, occupying the central position among the various techniques of click chemistry (JP-T-2006-502099, CHEMISTRY & CHEMICAL INDUSTRY Vol. 60-10, Oct. 2007). A method has also been reported wherein a nucleic acid is labeled by means of the click chemistry reaction, and cell cycle DNA synthesis is detected (WO2008/101024).

### SUMMARY OF THE INVENTION

### Problems to be Solved by the Invention

In WO2008/101024, no suggestion is given for application to the quantification of UDS, a form of DNA synthesis concomitant to DNA repair, since UDS is much less active than cell cycle DNA synthesis. Additionally, no report is available on application to RNA synthesis. In the prevention and/or treatment of DNA repair deficiency diseases and the amelioration of influences of DNA damage on living organisms, such as cell aging and carcinogenesis, it is essential to develop a substance that effectively promotes the repair of damaged DNA or suppresses the occurrence of DNA damage, or to search for new substances that regulate the activity of DNA repair synthesis by identifying novel genes involved in various DNA repairs. Accordingly, it is an object of the present invention to provide a method of quickly evaluating the DNA repair synthesis or inhibition of RNA synthesis as a substitute for currently available methods of UDS or RRS assay.

### Means for Solving the Problems

The present inventors extensively investigated with the aim of accomplishing the above-described object, and found that the addition cyclization reaction that occurs between a nucleotide getting incorporated at the time of DNA repair and a dye that detects the nucleotide enables a measurement of UDS in DNA repair processes, including NER. More specifically, the present inventors found that the use of 5-ethynyl-2′-deoxyuridine (EdU), an alkyne-coupled nucleoside analogue of thymidine, as a substitute for radioactive thymidine or BrdU, in the process of UDS assay, enables more convenient procedures, without using a radioisotope, and that it is hence possible to perform screening for damaged DNA repair substances or genes involved in DNA repair synthesis and the like based on UDS assay, with shortened time requirements of about half a day for UDS assay or clinical diagnosis of DNA repair deficiency diseases such as XP, while maintaining levels of sensitivity and accuracy comparable to those of the conventional art. Another finding was that by changing the saccharide that constitutes the nucleoside-like compound used in the determination of DNA repair synthesis activity from deoxyribose to ribose, inhibition of RNA synthesis can also be measured. Further investigations based on these findings have led to the development of the present invention.

Accordingly, the present invention provides:

[1] A screening method for a substance or gene that potentiates the DNA repair capability, a substance or gene that suppresses the induction of DNA damage in DNA-damaged cells, a substance or gene that suppresses the induction of DNA damage, a substance or gene that influences DNA repair, or the presence or absence of a toxicity of a substance with DNA damage as an index, comprising using a reagent set of a terminal alkyne-modified nucleoside derivative and a reporter molecule containing an azide moiety in combination, or a reagent set of an azide-modified nucleoside derivative and a reporter molecule containing a terminal alkyne in combination.
[2] The screening method according to [1] above, comprising the following steps (a), (b), (c) and (d):
(a) the step of treating cells with ultraviolet or a mutagen to induce DNA damage,
(b) the step of bringing into contact with each other a test substance, the cells treated in the step (a), and the terminal alkyne-modified nucleoside derivative,
(c) the step of measuring the incorporation of the terminal alkyne-modified nucleoside derivative in the cells after completion of the above-described steps using the reporter molecule containing an azide moiety, and comparing the incorporation with a control group, and
(d) the step of selecting a substance that alters the terminal alkyne-modified nucleoside derivative incorporation rate on the basis of the results of the comparison in the step (c) above.
[3] The screening method according to [1] above, comprising the following steps (a), (b′), (c′) and (d′):
(a) the step of treating cells with ultraviolet or a mutagen to induce DNA damage,
(b′) the step of bringing into contact with each other a test substance, the cells treated in the step (a), and the azide-modified nucleoside derivative,
(c′) the step of measuring the incorporation of the azide-modified nucleoside derivative in the cells after completion of the above-described steps using the reporter molecule containing a terminal alkyne, and comparing the incorporation with a control group, and
(d′) the step of selecting a substance that alters the azide-modified nucleoside derivative incorporation rate on the basis of the results of the comparison in the step (c′) above.
[4] The screening method according to [2] above, wherein the contact of the cell and test substance takes place before the step (a), the contact of the test substance is completed in the step (a), and the contact of the test substance is skipped in the step (b).
[5] The screening method according to [3] above, wherein the contact of the cells and test substance takes place before the step (a), the contact of the test substance is completed in the step (a), and the contact of the test substance is skipped in the step (b′).
[6] The screening method according to [2] above, wherein the operation to restrict the expression of a gene in the cells used in the step (a) takes place before the step (a), the expression of the gene in the cells is restricted in the step (a),
the contact of the test substance is skipped in the step (b), and
a gene that alters the incorporation rate with restricted expression is selected in the step (d).
[7] The screening method according to [3] above, wherein the operation to restrict the expression of a gene in the cells used in the step (a) takes place before the step (a),
the expression of the gene in the cells is restricted in the step (a),
the contact of the test substance is skipped in the step (b′), and
a gene that alters the incorporation rate with restricted expression is selected in the step (d′).
[8] The screening method according to [6] above, wherein the method is for searching for a gene that potentiates DNA repair capability in DNA-damaged cells, a gene that suppresses the induction of DNA damage, or a gene that influences DNA repair.
[9] The screening method according to [7] above, wherein the method is for searching for a gene that potentiates DNA repair capability in DNA-damaged cells, a gene that suppresses the induction of DNA damage, or a gene that influences DNA repair.
[10] The screening method according to [1] above, wherein the method is for searching for an active ingredient for a therapeutic agent for a disease accompanied by a DNA repair deficiency or a disease caused by a DNA repair deficiency that has occurred spontaneously in normal humans.
[11] The screening method according to [10] above, wherein the disease accompanied by a DNA repair deficiency is xeroderma pigmentosum, Cockayne's syndrome or trichothiodystrophy.
[12] The screening method according to [1] above, wherein the method is for searching for an ingredient of a cosmetic or pharmaceutical having an anti-aging effect.
[13] The screening method according to [12] above, wherein the cosmetic or pharmaceutical having an anti-aging effect is an ultraviolet-guard cosmetic.
[14] The screening method according to [1] above, wherein the method is for screening for the presence or absence of a toxicity of a substance with DNA damage as an index, comprising the following steps (b″), (c″) and (d″):
(b″) the step of bringing into contact with each other a test substance, cells, and the terminal alkyne-modified nucleoside derivative,
(c″) the step of measuring the incorporation of the terminal alkyne-modified nucleoside derivative in the cells contacted with the test substance, using the reporter molecule containing an azide moiety, and comparing this incorporation with the incorporation in control cells not contacted with the test substance, and
(d″) the step of judging a substance that raises the terminal alkyne-modified nucleoside derivative incorporation rate significantly as a substance that exhibits a cytotoxicity on the basis of the results of the comparison in the step (c″) above.
[15] The screening method according to [1] above, wherein the method is for screening for the presence or absence of a toxicity of a substance with DNA damage as an index, comprising the following steps (b′″), (c′″) and (d′″): (b′″) the step of bringing into contact with each other a test substance, cells, and the azide-modified nucleoside derivative, (c′″) the step of measuring the incorporation of the azide-modified nucleoside derivative in the cells contacted with the test substance, using the reporter molecule containing a terminal alkyne, and comparing this incorporation with the incorporation in control cells not contacted with the test substance, and
(d′″) the step of judging a substance that raises the azide-modified nucleoside derivative incorporation rate significantly as a substance that exhibits a cytotoxicity on the basis of the results of the comparison in the step (c′″) above.
[16] A diagnostic method for cells with a DNA repair deficiency, comprising the following steps (A), (B), (C) and (D):
(A) the step of treating cells from a subject with ultraviolet or a mutagen to induce DNA damage,
(B) the step of bringing into contact with each other the cells treated in the step (A) and a terminal alkyne-modified nucleoside derivative,
(C) the step of measuring the incorporation of the terminal alkyne-modified nucleoside derivative in ultraviolet-treated cells, using a reporter molecule containing an azide moiety, and comparing this incorporation with the incorporation in control cells treated to induce DNA damage, and
(D) the step of determining the presence or absence of a DNA repair deficiency in the subject on the basis of the results of the comparison in the step (C) above.
[17] A diagnostic method for cells with a DNA repair deficiency, comprising the following steps (A), (B′), (C′) and (D′):
(A) the step of treating cells from a subject with ultraviolet or a mutagen to induce DNA damage,
(B′) the step of bringing into contact with each other the cells treated in the step (A) and an azide-modified nucleoside derivative,
(C′) the step of measuring the incorporation of the azide-modified nucleoside derivative in ultraviolet-treated cells, using a reporter molecule containing a terminal alkyne, and comparing this incorporation with the incorporation in control cells treated to induce DNA damage, and
(D′) the step of determining the presence or absence of a DNA repair deficiency in the subject on the basis of the results of the comparison in other step (C′) above.
[18] The diagnostic method according to [16] above, wherein the cells with a DNA repair deficiency are derived from a patient possibly having xeroderma pigmentosum, Cockayne's syndrome or trichothiodystrophy.
[19] The diagnostic method according to [17] above, wherein the cells with a DNA repair deficiency are derived from a patient possibly having xeroderma pigmentosum, Cockayne's syndrome or trichothiodystrophy.
[20] A diagnostic kit for a disease accompanied by a DNA repair deficiency, comprising a reagent set of a terminal alkyne-modified nucleoside derivative and a reporter molecule containing an azide moiety in combination, or a reagent set of an azide-modified nucleoside derivative and a reporter molecule containing a terminal alkyne in combination.
[21] A screening kit for a substance or gene that potentiates repair capability in DNA-damaged cells, a substance or gene that suppresses the induction of DNA damage, a substance or gene that influences DNA repair, or the presence or absence of a toxicity of a substance with DNA damage as an index, comprising a reagent set of a terminal alkyne-modified nucleoside derivative and a reporter molecule containing an azide moiety in combination, or a reagent set of an azide-modified nucleoside derivative and a reporter molecule containing a terminal alkyne in combination.

### EFFECT OF THE INVENTION

The screening method of the present invention is based on a method of UDS assay that can be performed by highly simplified operating procedures without the use of a radioisotope and pretreatments such as DNA denaturation, and that has improved sensitivity, compared with conventional methods, as a result of the employment of a technique for detecting a nucleoside derivative having an alkyne bond at the terminal thereof using a detection reagent having an azide moiety (e.g., fluorescent dye), or a technique for detecting a nucleoside derivative having an azide moiety using a terminal alkyne-modified detection reagent (e.g., fluorescent dye). The screening method of the present invention is also effective in dramatically shortening the time taken for screening for a desired substance or gene. According to the diagnostic method of the present invention, the presence or absence of a DNA repair deficiency/DNA repair activity in test cells can be determined quickly. Hence, because an established diagnosis can be obtained early, it is possible to mitigate, or delay the progression of, symptoms in DNA repair deficiency diseases such as XP, for which no therapy has yet been established, by protection against sunlight and the like.

### BRIEF DESCRIPTION OF THE DRAWINGS

FIG. 1A shows measurements of ultraviolet-induced UDS activity in normal human primary fibroblasts. FIG. 1B shows typical photographs of EdU assay. FIGS. 1C-F show measurements of UDS activity as indicated by EdU incorporation (FIGS. 10 and D) or BrdU incorporation (FIGS. 1E and F).

FIGS. 2A-G show ultraviolet-induced UDS levels in NER normal cells and NER-deficient cells. FIG. 2H shows an UDS assay by ³H-thymidine incorporation. FIG. 2I shows typical photographs of EdU incorporation corresponding to FIGS. 2A-G.

FIG. 3A shows photographs of ultraviolet-induced EdU incorporation in normal 48BR fibroblasts and XPG-deficient XP20BE fibroblasts. FIG. 3B shows photographs of ultraviolet-induced EdU incorporation in quiescent cells.

FIG. 4A shows typical photographs of an EdU assay in the presence of latex beads. FIGS. 4B-D show histograms of UDS assays of the internal standard 48BR with 48BR (FIG. 4B), with XP12BR (FIG. 4C), and with XP15BR (FIG. 4D).

FIG. 5 shows improvement of sensitivity by extension of incubation time and acid extraction with the addition of FdU.

FIG. 6 shows examples of screening for substances by EdU incorporation.

### DESCRIPTION OF EMBODIMENTS

### I. Reagent Sets

The screening method and diagnostic method of the present invention comprise using a reagent set (1) of a terminal alkyne-modified nucleoside derivative and a reporter molecule containing an azide moiety in combination, or a reagent set (2) of an azide-modified nucleoside derivative and a reporter molecule containing a terminal alkyne in combination.

### Reagent set (1)

The terminal alkyne-modified nucleoside derivative contained in the reagent set (1) includes nucleosides having an alkynyl (preferably ethynyl) group at the terminus thereof; examples include, but are not limited to, ethynyldeoxyadenosine (EdA), ethynyldeoxyguanosine (EdG), ethynyldeoxycytidine (EdC), ethynylthymidine (EdT) and ethynyldeoxyuridine (EdU) for UDS assay, and ethynyladenosine (EA), ethynylguanosine (EG), ethynylcytidine (EC), ethynyluridine (EU) and the like for RRS assay. The terminal alkyne-modified nucleoside derivative, depending on the application, may be a nucleotide containing one of the above-described nucleoside moieties, for example, ethynyldeoxyadenosine triphosphate (E-dATP) and the like. These compounds can be produced by publicly known methods (Synlett, 2000, 1: pp. 86-88). In the present invention, commercial products can suitably be utilized.

In the present invention, it is preferable that in UDS assay, at least one kind of terminal alkyne-modified nucleoside derivative selected from the group consisting of EdA, EdG, EdC, EdT and EdU be used, with greater preference given to EdU. In RRS assay, it is preferable that at least one kind of terminal alkyne-modified nucleotide derivative selected from the group consisting of EA, EG, EC, and EU be used, with greater preference given to EU.

The reporter molecule containing an azide moiety, contained in the reagent set (1) includes molecules that bind to the above-described terminal alkyne-modified nucleoside derivative by the azide-alkyne cyclo-addition reaction (CuAAC), which is catalyzed by copper ions, and that can be detected. From the viewpoint of the ease of detection, it is preferable that the reporter molecule be a fluorescent dye; examples include Alexa Fluor (registered trademark) 488 azide, Alexa Fluor (registered trademark) 594 azide, Alexa Fluor (registered trademark) 647 azide, Oregon Green (registered trademark) 488 azide, tetramethylrhodamine azide and the like. These compounds can be produced by publicly known methods. In the present invention, commercial products can suitably be used.

### Reagent Set (2)

The azide-modified nucleoside derivative contained in the reagent set (2) includes azidated nucleosides; examples include, but are not limited to, azidodeoxyadenosine, azidodeoxyguanosine, azidodeoxycytidine, azidothymidine, azidodeoxyuridine and the like for UDS assay, and azidoadenosine, azidoguanosine, azidocytidine, azidouridine and the like for RRS assay. The azide-modified nucleoside derivative may be a nucleotide containing one of the above-described nucleoside moieties, for example, azidodeoxyadenosine triphosphate (N₃-dATP), and the like. These compounds can be produced by publicly known methods (Anal. Biochem., 1998, 258: pp. 195-201). In the present invention, commercial products can suitably be utilized.

In the present invention, it is preferable that in UDS assay, at least one kind of azide-modified nucleoside derivative selected from the group consisting of azidodeoxyadenosine, azidodeoxyguanosine, azidodeoxycytidine, azidodeoxythymine and azidodeoxyuridine be used, with greater preference given to azidodeoxyuridine. In RRS assay, it is preferable that at least one kind of terminal alkyne-modified nucleotide derivative selected from the group consisting of azidoadenosine, azidoguanosine, azidocytidine and azidouridine be used, with greater preference given to azidouridine.

The reporter molecule containing a terminal alkyne, contained in the reagent set (2) includes molecules that bind to the above described azide-modified nucleoside derivative by the azide-alkyne cyclo-addition reaction (CuAAC), which is catalyzed by copper ions, and that can be detected. From the viewpoint of the ease of detection, it is preferable that the reporter molecule be a fluorescent dye; examples include Alexa Fluor (registered trademark) 488 alkyne, Alexa Fluor (registered trademark) 594 alkyne, Alexa Fluor (registered trademark) 647 alkyne, Oregon Green (registered trademark) 488 alkyne, tetramethylrhodamine alkyne and the like. These compounds can be produced by publicly known methods. In the present invention, commercial products can suitably be utilized.

### II. Screening Method for a Substance or Gene with Repair of Damaged DNA Repair or Inhibition of RNA Synthesis as an Index

In an embodiment of this screening method, the reagent set (1) is used.

(a) Step of Treating Cells with Ultraviolet or a Mutagen to Induce DNA Damage

The choice of cells used in the step (a) is not particularly limited; cells derived from any organism or any tissue are acceptable. For search for a substance that is useful to humans, human-derived cells or cells of a disease model mouse and the like are preferable, and the cells may be primary culture cells or cells of a cell line in subculture. Examples include, but are not limited to, human primary fibroblasts such as normal 1BR, 48BR, 142BR and 251BR, XP-patient-derived cells such as XP12BR, XP15BR, XP13BR and XP20BE. These cells used may be those collected from animal tissue by a conventional method, or those of a cell line provided by a depository organization or a commercially available cell line. When the subject of screening is a gene, test cells treated by RNA interference, gene disruption, or another technique to restrict the expression of the screening subject gene are used.

A person skilled in the art can set cell culture conditions according to the choice of cells used; culture conditions for animal cells include a minimum essential medium (MEM), Dulbecco's modified Eagle medium (DMEM), RPMI1640 medium and 199 medium, each containing about 5 to 20% fetal bovine serum, and the like. Culture conditions are also likewise determined as appropriate. For example, the pH of the medium is normally about 6 to about 8, culture temperature is normally about 30 to about 40° C., and the cells are preferably maintained at a semi-confluent density.

A person skilled in the art can set conditions for ultraviolet treatment of cells according to the choice of cells used. For example, human fibroblasts are irradiated with ultraviolet light of 254 nm wavelength at 5 to 20 J/m², whereby DNA damage is induced.

A person skilled in the art can set a method of treating cells with a mutagen according to the choice of cells and mutagen used. Useful mutagens for this purpose include, but are not limited to, nitroso compounds such as nitrosoamine and nitrosoguanosine; alkylating agents such as the ethylating agent N-ethyl-N-nitrosourea (ENU) and the methylating agent methyl ethanesulfonate (EMS); polycyclic aromatic hydrocarbons such as benzpyrene and chrysene; DNA intercalaters such as ethidium bromide; DNA crosslinking agents such as cisplatin and mitomycin C; active oxygen; and ionizing radiation. In toxicity tests, only the step of treating cells with a test substance is performed, with no separate damage-inducing treatment performed.

(b) Step of Bringing into Contact with Each Other a Test Substance, the Cells Treated in the Step (a), and a Terminal Alkyne-Modified Nucleoside Derivative

The test substance may be any commonly known substance or a novel substance; examples include nucleic acids, glucides, lipids, proteins, peptides, organic low molecular compounds, compound libraries prepared using combinatorial chemistry technology, random peptide libraries prepared by solid phase synthesis and/or the phage display method, or naturally occurring ingredients derived from microorganisms, animals, plants, marine organisms and the like. When the subject of screening is a gene, the separate step of contacting with the test substance can be skipped. In toxicity tests, the contact with the test substance has already been performed in the step (a).

Contact of the test substance and cells and contact of the terminal alkyne-modified nucleoside derivative and cells are performed in a culture medium. In the case of human fibroblasts, the contact is completed by, for example, culturing the cells in a DMEM containing about 5 to 20% fetal bovine serum, adding the test substance and terminal alkyne-modified nucleoside derivative at specified concentrations to the medium, and continuing to culture them at about 30 to about 40° C. for about 0.5 to about 72 hours. Timing of contact of the test substance and cells can be chosen as appropriate according to the type of screening to be performed. For example, when a DNA damage repair promoting effect is determined by UDS activity, the contact may be performed after the step (a); when a DNA damage induction suppressing effect is determined by UDS activity, the contact may be performed before the step (a); when a DNA damage repair promoting effect is determined by RRS activity, the contact may be performed 12 to 48 hours after the end of the step (a), and the like.

(c) Step of Measuring the Incorporation of the Terminal Alkyne-Modified Nucleoside Derivative in the Cells after Completion of the Above-Described Steps Using a Reporter Molecule Containing an Azide Moiety, and Comparing this Incorporation with the Incorporation in a Control Group.

The cells whose contact has been completed in the step (b) are fixed or permeabilized using a surfactant-containing formaldehyde solution and the like. The cells are then thoroughly washed, and the free form of the terminal alkyne-modified nucleoside derivative is removed, after which the terminal alkyne-modified nucleoside derivative incorporated in the cells is measured using a reporter molecule containing an azide moiety. The binding reaction for the terminal alkyne group and azide is carried out by incubation at room temperature in the presence of Cu ions. The coupled reporter molecule can be measured by a conventional method according to the choice of reporter. When the reporter molecule used is a fluorescent dye, the fluorescence of a particular wavelength emitted by the fluorescent dye can easily be measured by variously combining an excitation wavelength and a detection wavelength, so this is preferred. In the measurement of the fluorescence, cells are immobilized on cover glass and photographed using a fluorescence microscope equipped with a CCD camera; the captured images are processed and can be analyzed using software. S-phase cells are omitted from the subjects of measurement. Suspended cells can be analyzed by flowcytometry.

By culturing cells on a microtiter plate, high throughput screening can be achieved and analysis is possible using a high throughput imaging system such as In-Cell-Analyzer.

Cells serving for control are measured in the same manner, and the incorporation therein is compared with the incorporation of terminal alkyne-modified nucleoside derivative in the cells undergoing the test operation. Thus, UDS or RRS can be measured in this step.

(d) Step of Selecting a Substance or Gene that Alters the Terminal Alkyne-Modified Nucleoside Incorporation Rate on the Basis of the Results of the Comparison in the Step (c) Above

If the amount of terminal alkyne-modified nucleoside derivative incorporated in the cells undergoing the test operation changes significantly, compared with the amount of terminal alkyne-modified nucleoside derivative incorporated in the cells undergoing the control operation, the substance or gene being screened for in the test operation can be selected as a substance or gene that alters the terminal alkyne-modified nucleoside incorporation rate. The substance or gene thus selected can be utilized for a broad range of applications as a candidate substance or gene that potentiates DNA damage repair, suppresses the onset of DNA damage, or is involved in DNA repair. In toxicity tests, the substance or gene can be utilized for the evaluation of the toxicity of a subject substance.

In another embodiment of the screening method of the present invention, a reagent set (2) is used.

(a) Step of Treating Cells with Ultraviolet or a Mutagen to Induce DNA Damage

This is the same as the above-described step (a). When the subject of screening is a gene, test cells treated by RNA interference, gene disruption, or another technique to restrict the expression of the screening subject gene are used. In toxicity tests, only the step of treating cells with the test substance is performed, with no separate damage-inducing treatment performed.

(b′) Step of Bringing into Contact with Each Other a Test Substance, the Cells Treated in the Step (a), and an Azide-Modified Nucleoside Derivative

This is the same as the above-described step (b), except that an azide-modified nucleoside derivative is used in place of the terminal alkyne-modified nucleoside derivative used in the step (b). When the subject of screening is a gene, the separate step of contacting with the test substance can be skipped. In toxicity tests, contact with the test substance has already been achieved in the step (a).

(c′) Step of Measuring the Incorporation of the Azide-Modified Nucleoside Derivative in the Cells after Completion of the Above-Described Steps Using a Reporter Molecule Containing a Terminal Alkyne, and Comparing this Incorporation with the Incorporation in a Control Group.

This is the same as the above-described step (c), except that the cells whose contact has been completed in the above-described step (b′) are measured using a reporter molecule containing a terminal alkyne in place of the azide-modified reporter molecule used in the step (c).

(d′) Step of Selecting a Substance or Gene that Alters the Azide-Modified Nucleoside Derivative Incorporation Rate on the Basis of the Results of the Comparison in the Step (c′) Above

If the amount of azide-modified nucleoside derivative incorporated in the cells undergoing the test operation changes significantly, compared with the amount of azide-modified nucleoside derivative incorporated in the control cells not contacted with the test substance, the substance or gene subjected being screened for in the test operation can be selected as a substance or gene that alters the azide-modified nucleoside incorporation rate. The substance or gene thus selected can be utilized in a broad range of applications as a candidate substance or gene that potentiates DNA damage repair, suppresses the onset of DNA damage, or is involved in DNA repair. In toxicity tests, the substance or gene can be utilized for the evaluation of the toxicity of a subject substance.

The substances and genes selected by the screening method of the present invention are useful in developing active ingredients for therapeutic agents for diseases accompanied by a DNA repair deficiency, ingredients for cosmetics and/or pharmaceuticals having an anti-aging effect (e.g., ultraviolet-guard cosmetics), or new screening methods for these ingredients and the like (generating knockout mice wherein one of the selected genes is knocked out, and subjecting them to screening, and the like). As mentioned herein, “an anti-aging effect” refers to a suppressive action on cell aging due to ultraviolet irradiation, active oxygen and the like.

Diseases accompanied by a DNA repair deficiency include, but are not limited to, nucleotide excision repair deficiency diseases, for example, xeroderma pigmentosum, Cockayne's syndrome and trichothiodystrophy. Diseases caused by a nucleotide excision repair deficiency that has occurred spontaneously in normal humans include, but are not limited to, skin cancers and the like.

### III. Diagnostic Method for Cells with a DNA Repair Deficiency

The present invention provides a diagnostic method for cells with a DNA repair deficiency using the above-described reagent set (1) or (2). In the diagnostic method, it is possible to diagnose a disease accompanied by a DNA repair deficiency by detecting a cell with the DNA repair deficiency. In an embodiment of this diagnostic method, cells with a nucleotide excision repair deficiency are detected using the reagent set (1).

(A) Step of Treating Cells from a Subject with Ultraviolet or a Mutagen to Induce DNA Damage

Cells to be treated to induce DNA damage are prepared by collecting a portion of living tissue of the subject (skin biopsy and the like), and dispersing the cells by enzyme treatment to obtain primary culture cells. It is also preferable that normal skin fibroblasts, an XP-patient-derived fibroblast cell line [e.g., XP15BR (XP-A), XP20BE (XP-G), XP13BR (XP-C), XP12BR (XP-D)] for diagnosis of XP, and CS-patient derived fibroblasts [CS10LO (CS-B)] for diagnosis of CS, be prepared for control.

Example conditions of DNA damage treatment include irradiation of ultraviolet light of 254 nm wavelength to human fibroblasts at 5 to 20 J/m².

(B) Step of Bringing into Contact with Each Other the Cells Treated in the Step (A) and a Terminal Alkyne-Modified Nucleoside Derivative

This is the same as the step (b) in the screening method, except that no test substance is added in the step (b).

(C) Step of Measuring the Incorporation of the Terminal Alkyne-Modified Nucleoside Derivative in the Cells Treated to Induce DNA Damage, Using a Reporter Molecule Containing an Azide Moiety, and Comparing this Incorporation with the Incorporation in Control Cells Treated to Induce DNA Damage

This is the same as the step (c) in the screening method.

### (D) Step of Determining the Presence or Absence of a Nucleotide Excision Repair Deficiency in the Subject on the Basis of the Results of the Comparison in the Step (C) Above

If the incorporation of the terminal alkyne-modified nucleoside derivative in the cells from the subject is significantly lower than the incorporation in normal control cells, the subject can be judged to have a reduced nucleotide excision repair capability. Also, comparing the incorporation in the cells from the subject with, for example, the incorporation in each type of cells from typical XP patients, an index is obtained to determine whether or not the subject is suffering from any type of XP.

In another embodiment of this diagnostic method, a reagent set (2) is used.

(A) Step of Treating Cells from a Subject with Ultraviolet or a Mutagen to Induce DNA Damage

This is the same as the above-described step (A).

(B′) Step of Bringing into Contact with Each Other the Cells Treated in the Step (A) and an Azide-Modified Nucleoside derivative

This is the same as the above-described step (B), except that an azide-modified nucleoside derivative is used in place of the terminal alkyne-modified nucleoside derivative used in the step (B).

(C′) Step of Measuring the Incorporation of the Azide-Modified Nucleoside Derivative in the Cells Treated to Induce DNA Damage, Using a Reporter Molecule Containing a Terminal Alkyne, and Comparing this Incorporation with the Incorporation in Control Cells Treated to Induce DNA Damage

This is the same as the above-described step (C), except that the cells whose contact has been completed in the above-described step (B′) are measured using a reporter molecule containing a terminal alkyne in place of the azide-modified reporter molecule used in the step (C).

### (D′) Step of Determining the Presence or Absence of a Nucleotide Excision Repair Deficiency in the Subject on the Basis of the Results of the Comparison in the Step (C′) Above

This is the same as the above-described step (D), except that the incorporation of an azide-modified nucleoside derivative is tested in place of the incorporation of the terminal alkyne-modified nucleoside derivative used in the step (D).

The diagnostic method of the present invention enables a determination of whether a subject is suffering from any disease caused by a DNA repair deficiency, such as xeroderma pigmentosum, Cockayne's syndrome or trichothiodystrophy, by making a comparison with control cells from the disease. It is also possible to set reference values for the diagnosis by assay cells from these patients using the diagnostic method of the present invention.

The present invention provides a diagnostic kit for a disease accompanied by a DNA repair deficiency, comprising the above-described reagent set (1) or reagent set (2).

The present invention also provides a screening kit for a substance or gene that potentiates repair capability in DNA-damaged cells, a substance or gene that suppresses the induction of DNA damage, a substance or gene that influences DNA repair, or the presence or absence of a toxicity of a substance with DNA damage as an index, comprising the above-described reagent set (1) or reagent set (2).

The above-described kit may further comprise a solvent or reagent such as a catalyst (CuSO₄ and the like) for a click chemistry reaction, or a fluorescent dye for cell cycle detection, or an instruction document stating that the reagent set can be used, or should be used, for detection of unscheduled DNA synthesis in DNA-damaged cells.

### EXAMPLES

The present invention is hereinafter described in further detail by means of the following Examples.

### Test Example 1

### Optimization of Ultraviolet-Induced UDS by EdU Incorporation

48BR cells were cultured on cover glass, and maintained at a confluent density. The cells were washed with PBS, and subsequently irradiated with different doses (5-20 J/m²) of ultraviolet light (254 nm). Immediately after the irradiation, the cells were cultured in a serum-free DMEM containing 10 μM EdU for different lengths of periods (0.5, 1, 2 and 4 hours). The cells were then washed with PBS, and fixed and permeabilized in a PBS containing 2% formaldehyde, 0.5% Triton X-100 and 300 μM sucrose. After being thoroughly washed with PBS, the cells were treated with a PBS supplemented with 10%

FBS for 30 minutes. The EdU incorporated was detected by a fluorescent azide binding reaction (Click-It™, Invitrogen). The procedures for the detection are as follows: The cells were cultured along with azide-coupled Alexa Fluor 488 dye in a TBS supplemented with 4 mM CuSO₄ for 30 minutes, and then washed with a PBS containing 0.05% Tween-20 (PBST) three times. After the cover glass was immersed in PBS, the cells were fixed in a PBS supplemented with 3.7% formaldehyde for 20 minutes, and the cover glass was mounted on a glass slide using Aqua-Poly/Mount (Polysciences). The cells were photographed using a fluorescence microscope equipped with a CCD camera (BIOREVO9000-KEYENCE), and the images captured were analyzed using ImageJ software (NIH). At least 50 non-5-stage cells were randomly selected from each visual field captured, and mean nuclear fluorescence intensity was calculated. For control, EdU assay was performed under the same conditions, except that normal cells were mock-treated in the process of ultraviolet irradiation, and images were analyzed.

### (Results)

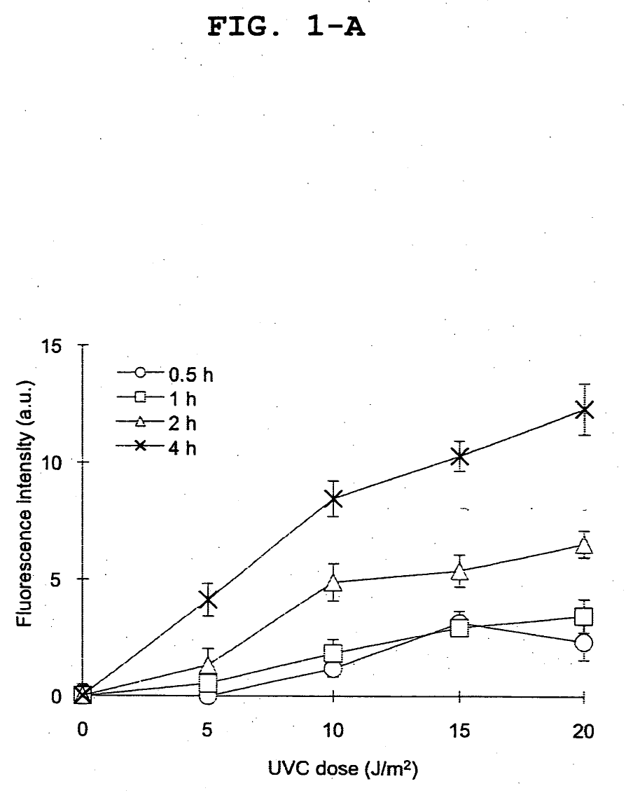

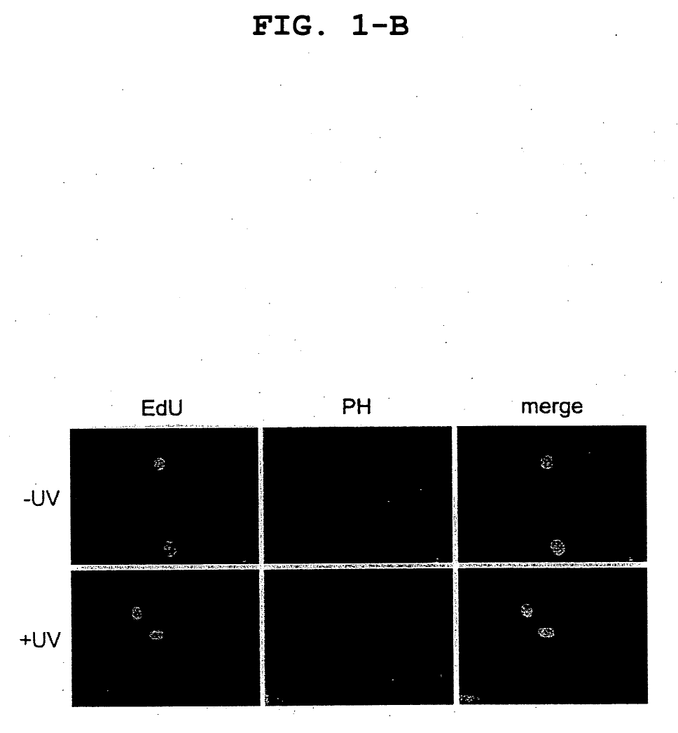

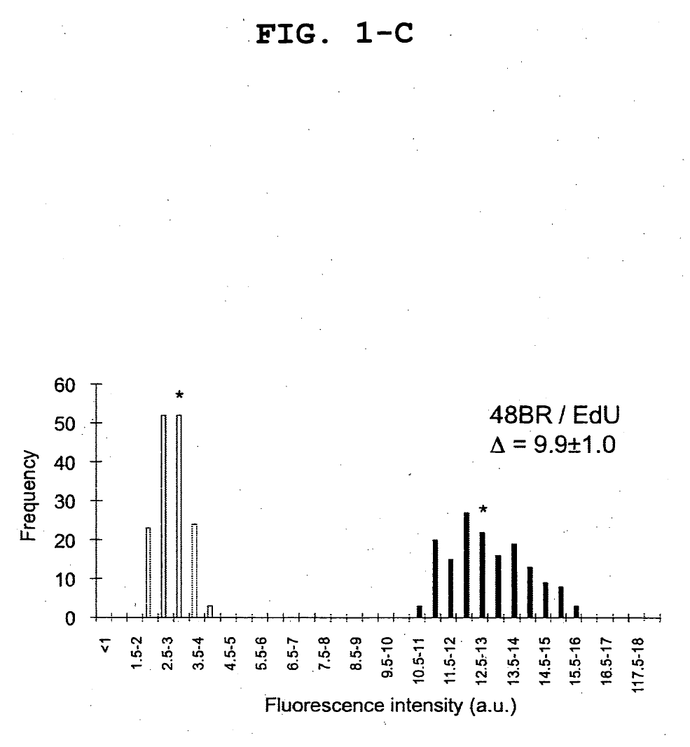

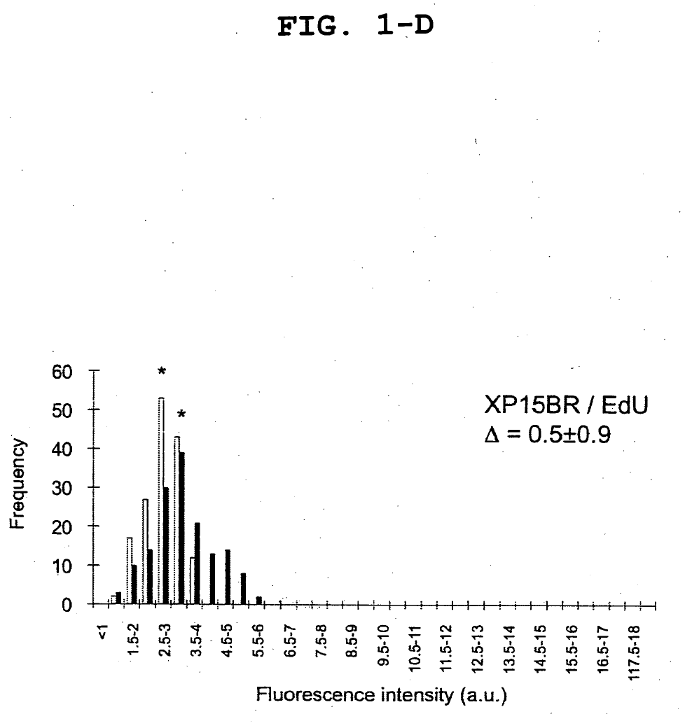

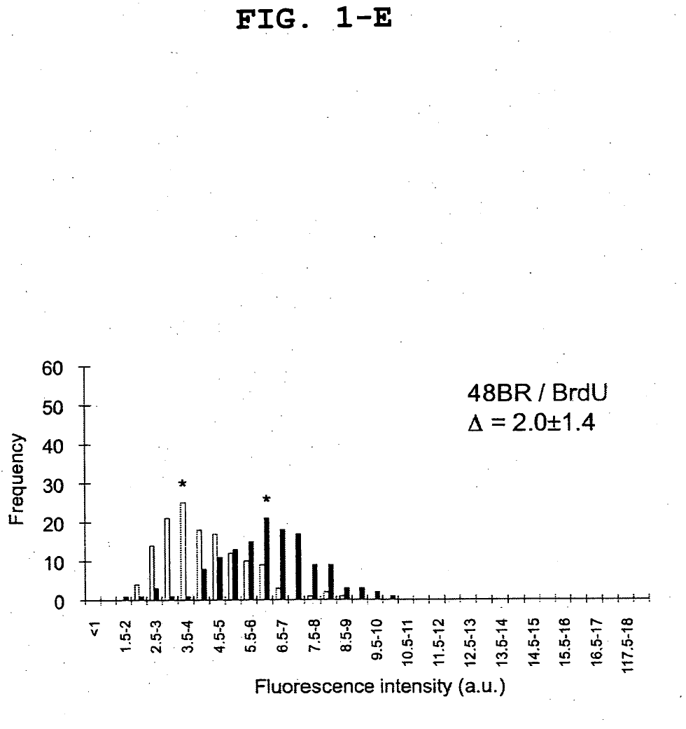

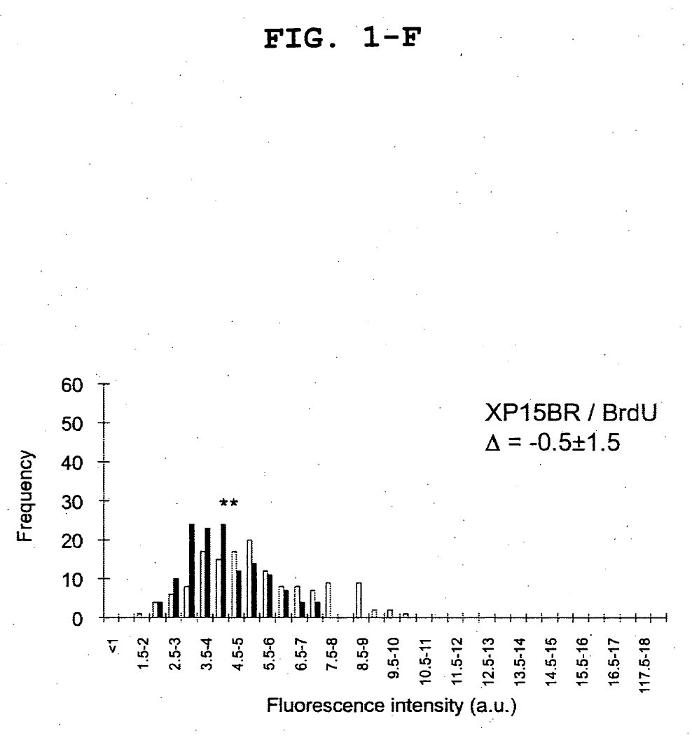

From FIG. 1A, it is evident that nuclear fluorescence intensity is proportional to both ultraviolet dose and EdU incubation time; it was also found that this parameter can be semi-quantitatively converted directly to UDS activity to indicate the amount of EdU incorporated. At relatively low doses of ultraviolet irradiation, nuclear fluorescence signals could be detected via EdU incubation for a short time after ultraviolet irradiation. It was also shown that incubation of cells along with EdU after 20 J/m² ultraviolet irradiation for 2 hours represents the optimum conditions for UDS assay. These conditions were used for all experiments that followed unless otherwise specified.

As shown in FIG. 1B, the strong signal from cell cycle DNA synthesis in S-phase cells observed with the use of ³H-thymidine-labeled autoradiography is distinguishable from the EdU incorporation by the weaker, cell cycle-non-dependent unscheduled DNA synthesis due to repair synthesis. Furthermore, nonspecific cytoplasm stains or DNA replication non-dependent nuclear signals were little detected; it was shown that the fluorescent azide binding reaction is specific for the EdU incorporated.

These findings suggest that this technique may have a sufficient sensitivity to detect trace amounts of UDS activity.

### Test Example 2

### Comparison of EdU Incorporation Assay and BrdU Incorporation Assay

In human-derived normal primary fibroblast 48BR cells and XP-A-patient-derived primary fibroblast XP15BR cells, EdU or BrdU was incorporated after ultraviolet irradiation (mock treatment for control), and UDS activity was compared.

The procedures for EdU treatment of the cells were as described above. In measuring BrdU incorporation, the cells, along with BrdU, were cultured and washed, followed by fixation and permeabilization, under the same conditions, except that 5 μM BrdU was added in place of EdU. After being thoroughly washed with PBS, the cells were treated with a PBS supplemented with 4 M HCl for 15 minutes to achieve DNA denaturation. For neutralization, the cells were thoroughly washed with PBS, and subsequently fixed in a PBS supplemented with 10% FBS for 30 minutes. After the cells, along with mouse anti-BrdU antibody (BD, diluted with PEST to 1:150 volume), were incubated for 1 hour, and the BrdU incorporated was detected. The cells were then washed with PBST three times, followed by incubation along with Alexa Fluor 488-coupled goat anti-mouse IgG (Invitrogen, diluted with PEST to 1:500 volume) for 1 hour. The cover glass was immersed in PBS, and the cells were fixed in a PBS supplemented with 3.7% formaldehyde for 20 minutes; the cover glass was mounted on a glass slide using Aqua-Poly/Mount (Polysciences). Images were captured and analyzed as described above.

### (Results)

On both normal 48BR fibroblasts (UDS-positive, FIG. 1C) and XP-deficient 15BR fibroblasts (UDS-negative, FIG. 1D), intensity variation plot from an UDS assay based on EdU incorporation is similar to an established plot from an autoradiography-based experiment. In this Test Example, however, the background UDS level was found to be higher than that from autoradiography. The EdU assay constantly yielded small SD (10-15%).

A comparison of the relative sensitivities of EdU-based assay and BrdU-based assay revealed that the sensitivity and resolution of BrdU are subject to limitations compared with EdU (FIGS. 1E and 1F). This is also suggested from the fact that BrdU has been reported to be used generally for S-phase labeling, whereas few reports are available on its use for UDS assay. The histograms of the normal 48BR fibroblasts and XP-deficient XP15BR fibroblasts can also be distinguished from each other by a BrdU-based assay (FIGS. 1E and 1F), but both the resolution and SD improved remarkably in the EdU method, showing that in UDS assay, higher sensitivity is obtained with Edu than with BrdU.

### Test Example 3

### Ultraviolet-Induced EdU Incorporation in NER-Deficient Primary Fibroblasts

To determine the applicability of the method of UDS assay based on EdU incorporation to the diagnosis of XP, ultraviolet-induced EdU incorporation levels were investigated in several kinds of NER-deficient primary fibroblasts and a normal control (no ultraviolet treatment for the control). The cells used were 48BR, 1BR, XP15BR, XP20BE, XP13BR, XP12BR, and CS10LO. The EdU-based UDS activity determination is as described above.

### (Results)

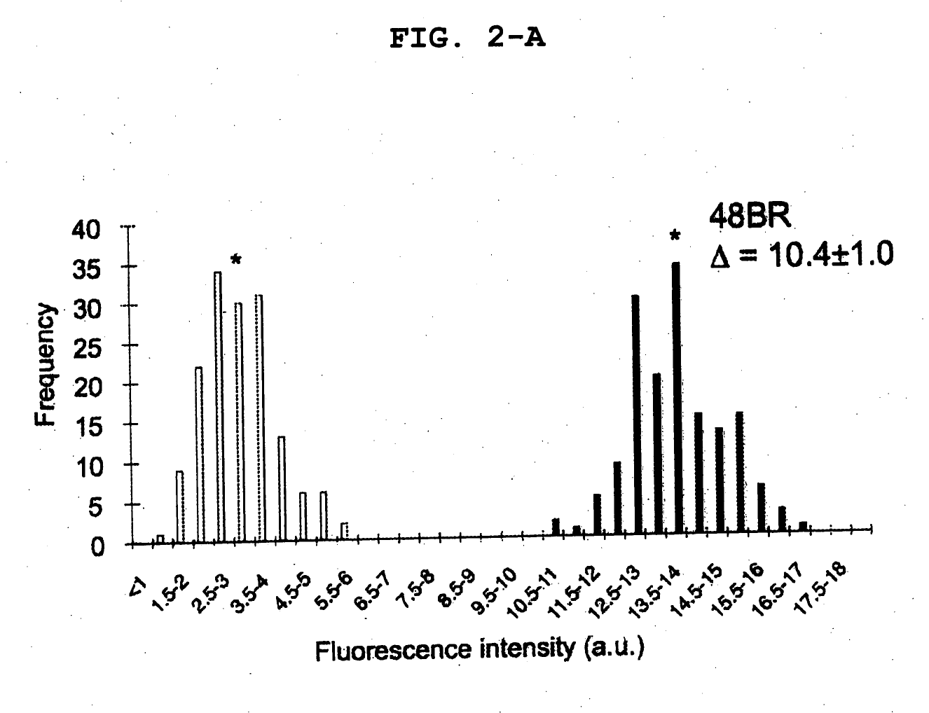

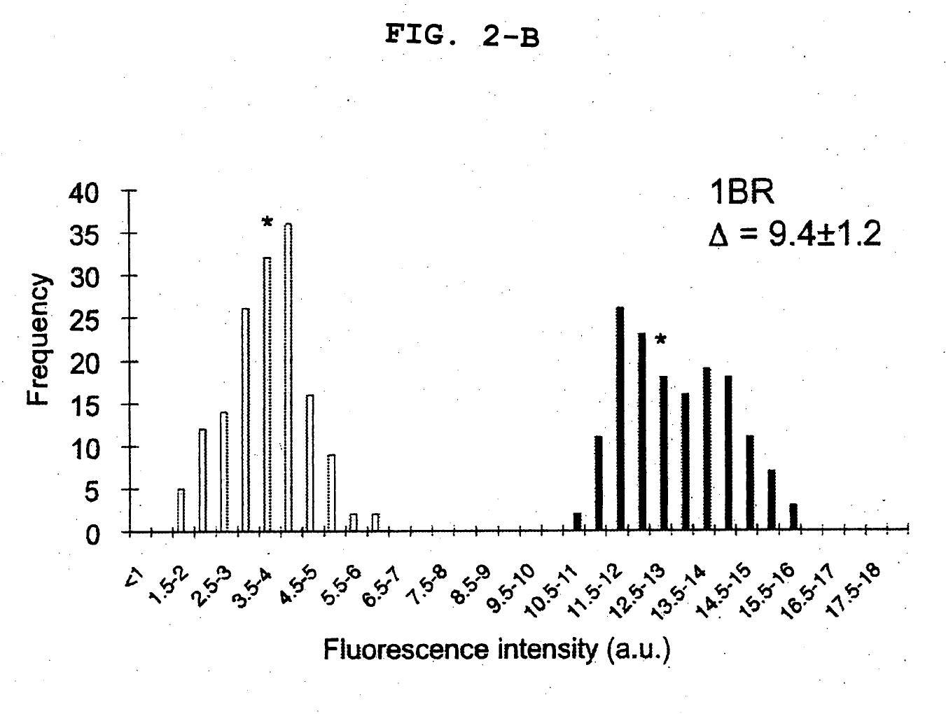

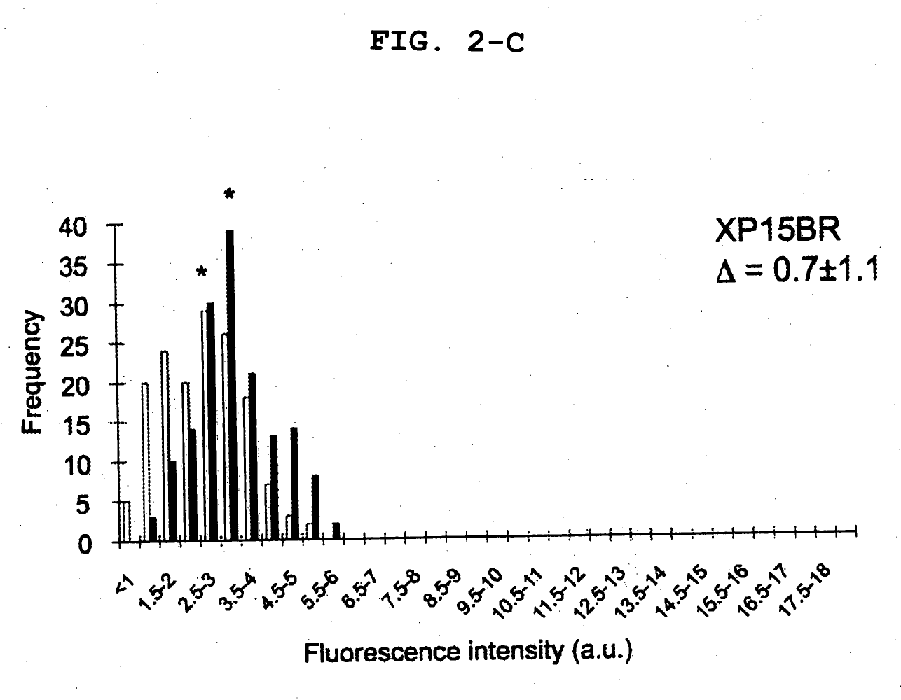

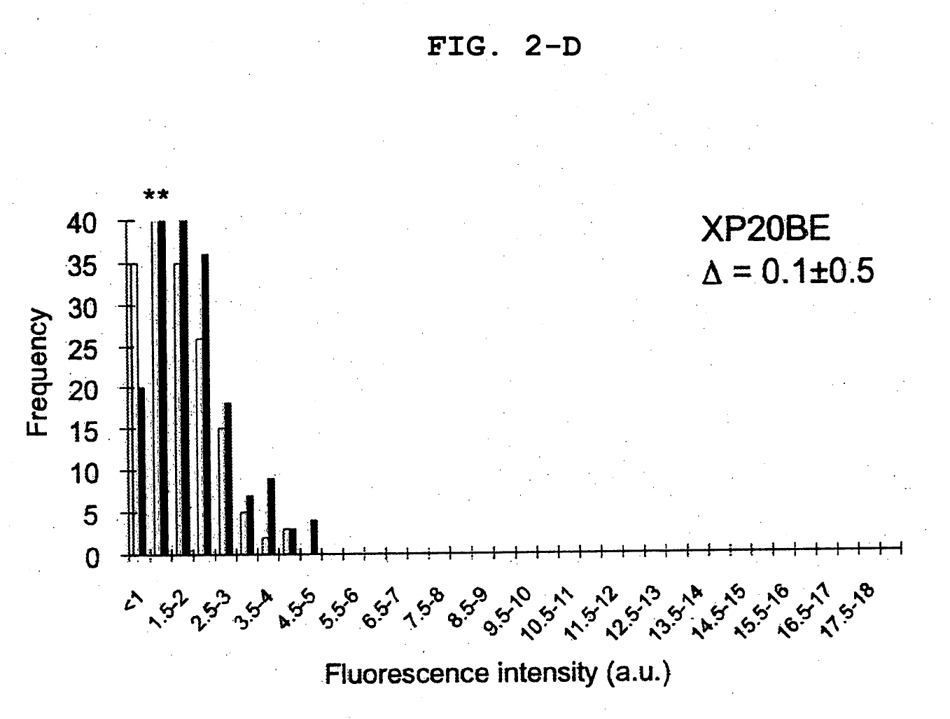

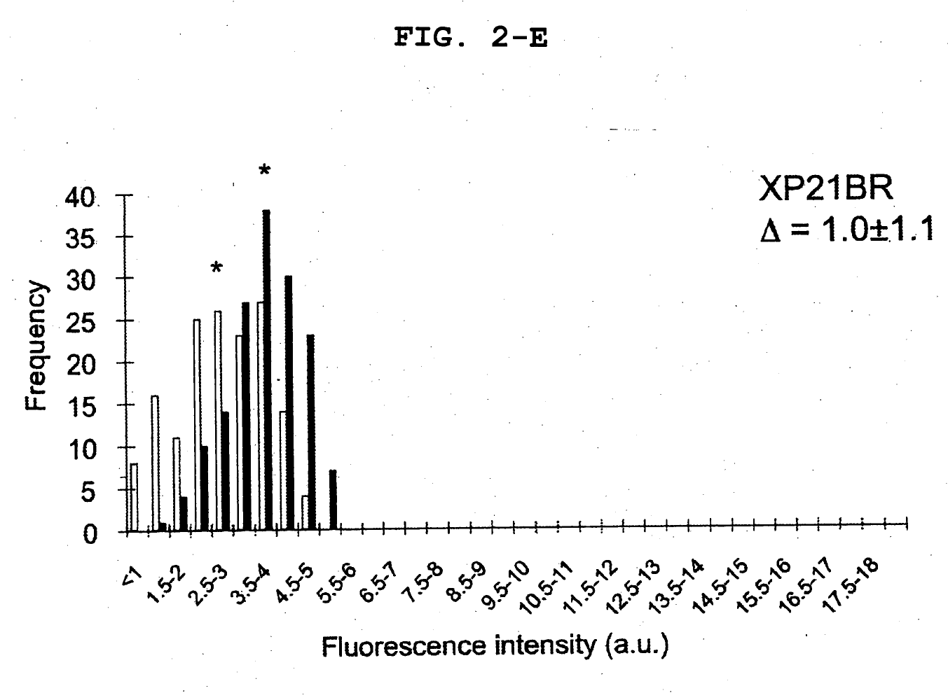

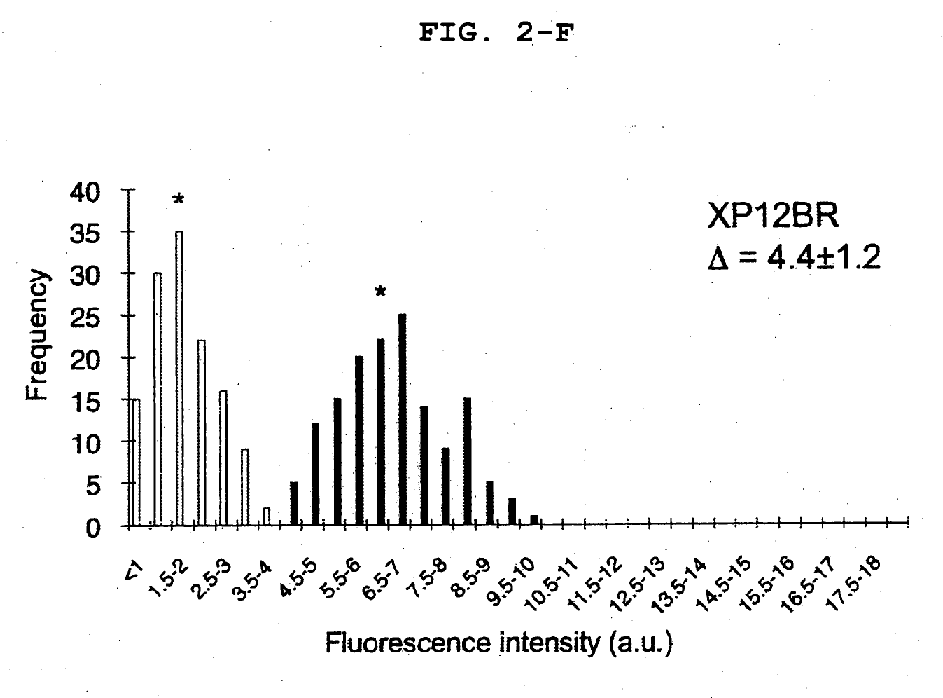

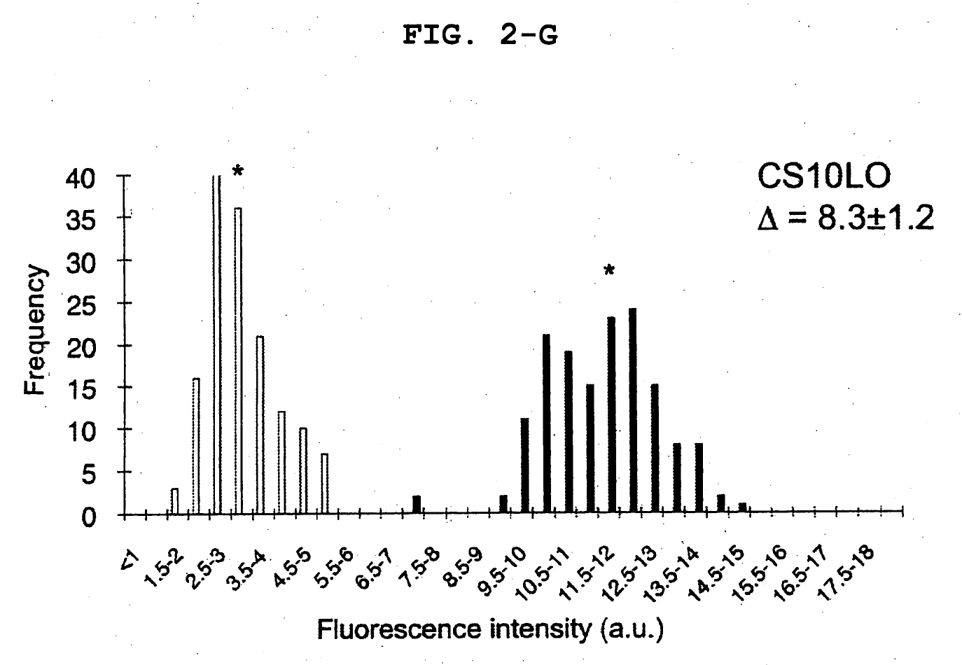

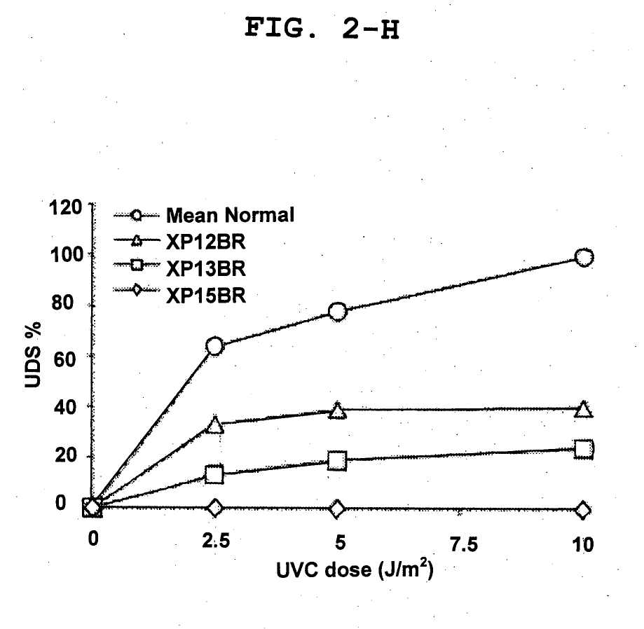

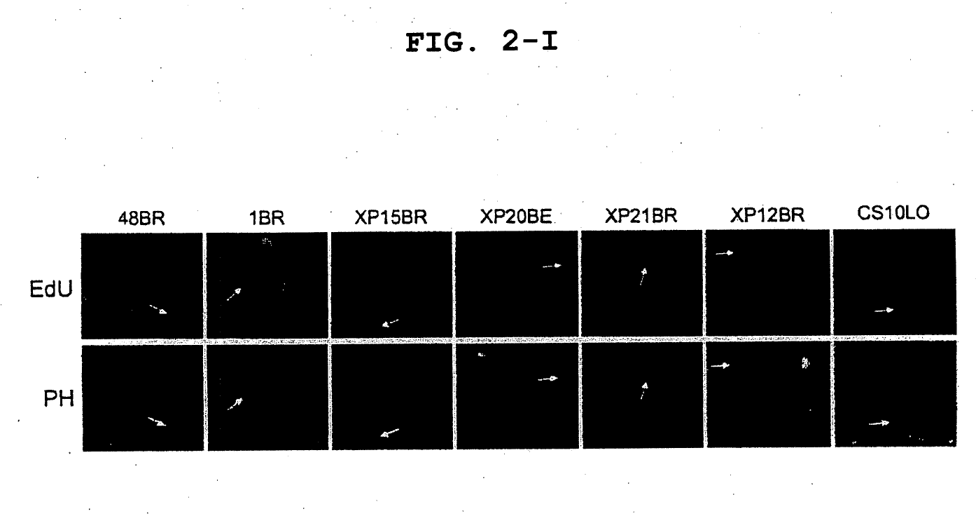

Test results are shown in the histograms in FIGS. 2A-G and the photographs in FIG. 2I. Because XP is a genetically heterogeneous disease (a disease involving more than one causal gene), both the NER gene having an abnormality and the type of mutation determine the extent of deficiency in UDS. UDS was measured in XP15BR (XP-A), XP20BE (XP-G/CS), XP13BR (XP-C), XP12BR (XP-D), and CS-patient-derived CS10LO (CS-B) cells. As a result of ultraviolet irradiation at 20 J/m², substantial UDS in normal fibroblasts 48BR (FIG. 2A) and 1BR (FIG. 2B) was observed. Although little UDS was detected in XP15BR fibroblasts from a severely affected XP patient (XP-A, FIG. 2C) and XP20BE (XP-G, FIG. 2D) fibroblasts, an UDS activity close to the limit of detection (up to 20% of normal values) was detected in a XP-C patient-derived XP13BR (FIG. 2E) fibroblasts. As shown in FIG. 2I, EdU incorporation occurred in normal cells, whereas EdU incorporation was not evident in NER-deficient cells; the ultraviolet-induced EdU incorporation in non-5-phase cells was shown to be specific for the DNA repair synthesis in NER.

### Comparative Example 1

### ³H-Thymidine Incorporation Assay

Details of the UDS assay based on ³H-thymidine are as follows. Stationary cells [XP15BR, XP13BR, XP12BR and four different normal cell lines (1BR, 48BR, 142BR and 251BR)] were cultured in 1% DMEM for 3 days (3×10⁵ cells/plate of 5 cm diameter). After incubation along with 10 mM hydroxyurea (HU) for 1 hour, the cells were irradiated with ultraviolet at the indicated dose. The cells were further incubated with a 1% DMEM containing 10 μCi/ml ³H-thymidine and 10 mM HU for 3 hours. The ³H-thymidine incorporated in acid-insoluble substance was measured by liquid scintillation counting.

The UDS levels in XP15BR, XP13BR, XP12BR and four different normal cells (1BR, 48BR, 142BR and 251BR) (for normal cells, the mean of the levels for the 4 cell lines) were compared with those obtained using EdU incorporation. The UDS levels were normalized, and calculated as ratios of UDS in normal cells treated at 10 J/m². The Mean Normal in FIG. 2H is the mean UDS level for the four different normal cell lines (1BR, 48BR, 142BR and 251BR).

### (Results)

As shown in FIG. 2H, little UDS activity was detected in XP15BR, whereas XP13BR and XP12BR exhibited UDS activity levels of up to about 20% and up to about 40% of normal values, respectively; this agrees well with the results of the EdU-based assay in FIGS. 2C, 2E and 2F. This result also demonstrates that cells exhibiting intermediate UDS activity (20 to 40% of normal UDS level) can be distinguished from both cells with normal UDS activity and those with severe UDS deficiency by an assay based on EdU incorporation.

Meanwhile, FIG. 2G shows that the UDS level lowered slightly (up to 20%) compared with normal fibroblasts; however, it cannot immediately be concluded that this is a significant reduction and tends to decrease in the range of NER abnormalities. Because CS is a form of NER that involves no more than a functional reduction of the TCR pathway, also because GGR, which accounts for about 90% of total NER activity, is normal, the NER deficiency observed is often minute.

### Comparative Example 2

### Comparison of Compatibility with Immunological Staining

To determine the applicability of the UDS assay based on EdU incorporation to clinical diagnosis, the compatibility of this technique with UDS assays by other standard techniques using an internal standard was evaluated. 48BR cells and XP20BE cells were cultured in a 1:1 mixture (48BR alone for ki67 staining). Ultraviolet irradiation, EdU incubation and fixation-permeabilization steps were performed as described above. For antibody detection, the cells were fixed with 10% FBS in PBS for 30 minutes, followed by incubation along with mouse monoclonal anti-XPG antibody (8H7, Santa Cruz Biotechnology) (diluted with PBST to 1:100 volume) or rabbit monoclonal anti-ki67 antibody (SP6, Thermo Scientific) (diluted with PBST to 1:100 volume) for 1 hour. Subsequently, the cells were washed with PBST three times, followed by incubation along with each secondary antibody [Alexa Fluor 594-coupled goat anti-mouse IgG antibody (Invitrogen, diluted with PBST to 1:1000 volume) and Alexa Fluor 594-coupled goat anti-rabbit IgG antibody (Invitrogen, diluted with PBST to 1:1000 volume) for detection of XPG and ki67, respectively] for 1 hour. To avoid unwanted nuclear fluorescence, DAPI staining was skipped. After being thoroughly washed with PBST, the cells were fixed in a PBS supplemented with 3.7% formaldehyde for 20 minutes. Subsequently, EdU detection was performed as described above.

### (Results)

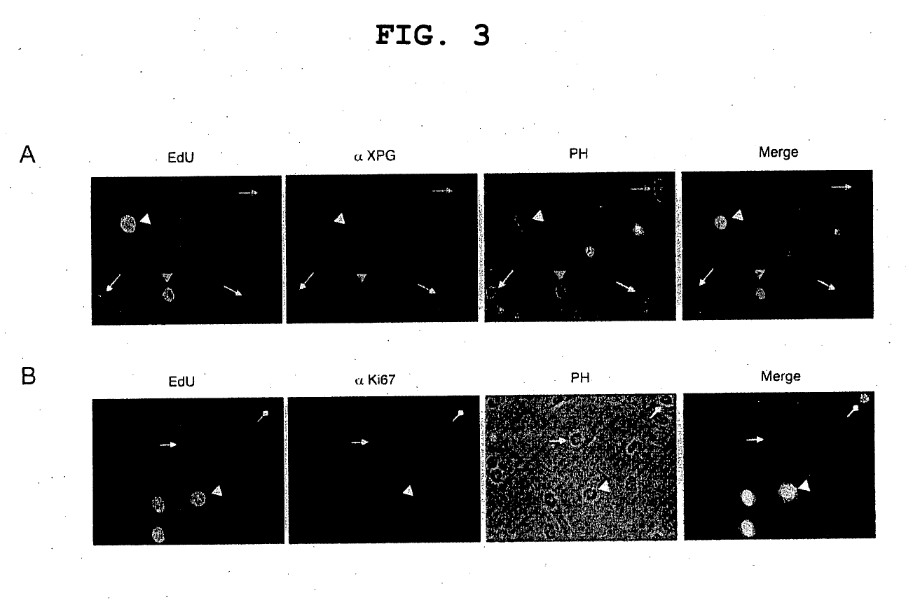

FIGS. 3A and 3B demonstrate that fluorescent azide binding to EdU is completely compatible with immunofluorescent stains. When index fibroblasts and the target in this Test Example were co-cultured on the same cover glass, a strict internal control was obtained. Referring to FIG. 3A, normal fibroblasts and XPG-deficient fibroblasts were co-cultured, followed by an UV-UDS assay in combination with immunological staining with XPG antibody; EdU-positive cells (except for S phase) agreed completely with XPG-positive cells, demonstrating that the two different cell populations can be distinguished in the UDS assay. Likewise, referring to FIG. 3B, the EdU assay was examined for cell cycle selectivity; it was demonstrated that co-immunological staining with proliferation marker ki67 is capable of detecting UDS in quiescent fibroblasts (no ki67 staining) and a population of proliferated fibroblasts.

### Test Example 4

From the results described above, it is evident that the level of NER deficiency in UDS activity is variable among different XP patients. Ideally, whether or not the patient is NER-deficient is determined by comparing the residual UDS activity in fibroblasts from the patient and that in index cells. To confirm this, normal fibroblasts, previously treated with latex beads introduced into the cytoplasms thereof, along with patient-derived fibroblasts, were co-cultured in practical UDS-based XP diagnosis. “A bead-labeled cell” provides an internal standard for eliminating sample-to-sample variation in staining profile, that can be detected under a phase-contrast microscope. Another test was conducted to determine the compatibility of this technique with EdU assay. First, examination was performed to determine whether or not latex beads act on nuclear fluorescence intensity.

### EdU Assay in Other Presence of Latex Beads

Normal 48BR cells were pre-labeled with latex beads 0.5 μm in diameter, and co-cultured with indicator XP fibroblasts (48BR, XP12BR, XP15BR: no beads) on cover glass. The cells were then irradiated with ultraviolet light (20 J/m²), followed by incubation along with 10 μM EdU for 2 hours. Subsequently, the cover glass was treated in the same manner as Comparative Example 1.

### (Results)

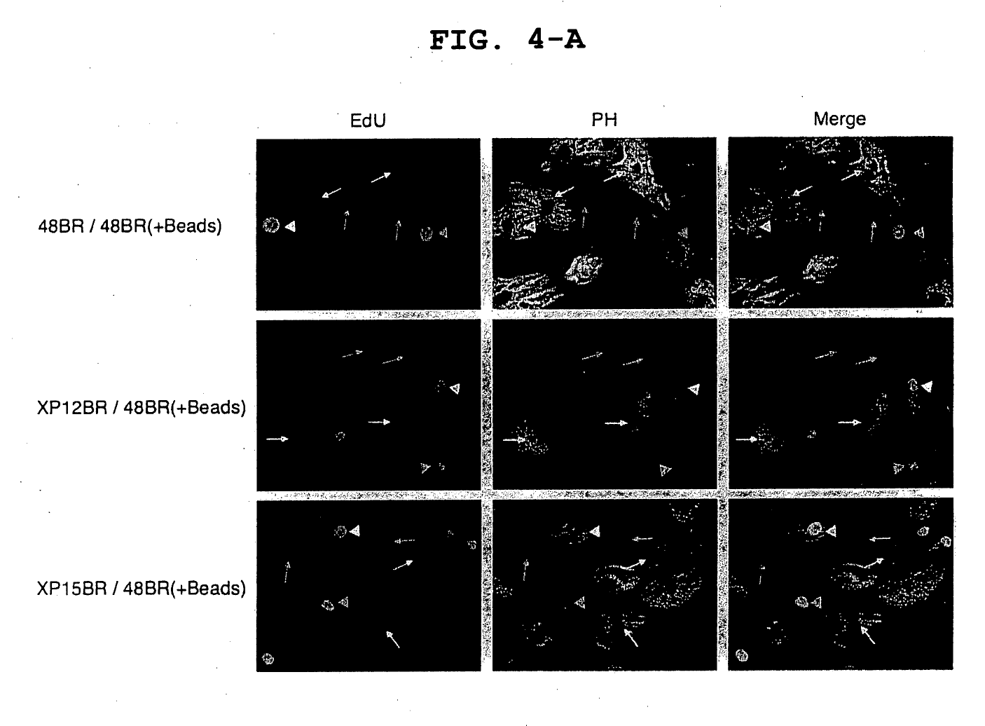

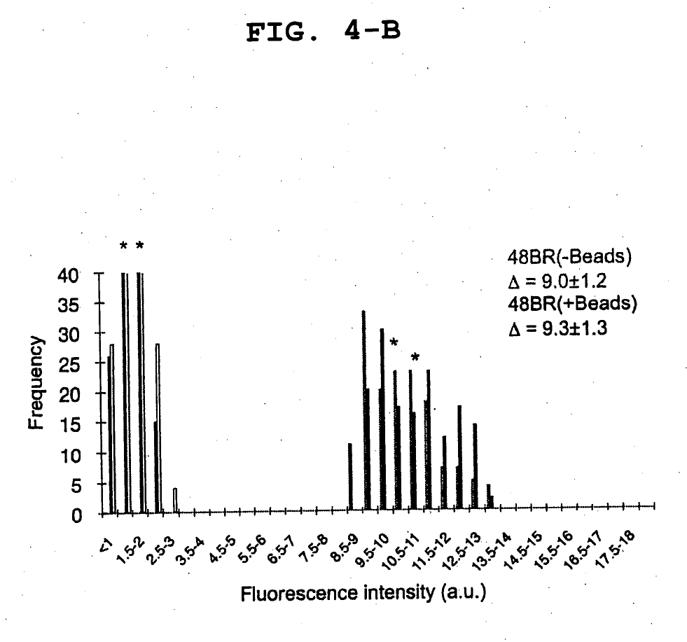

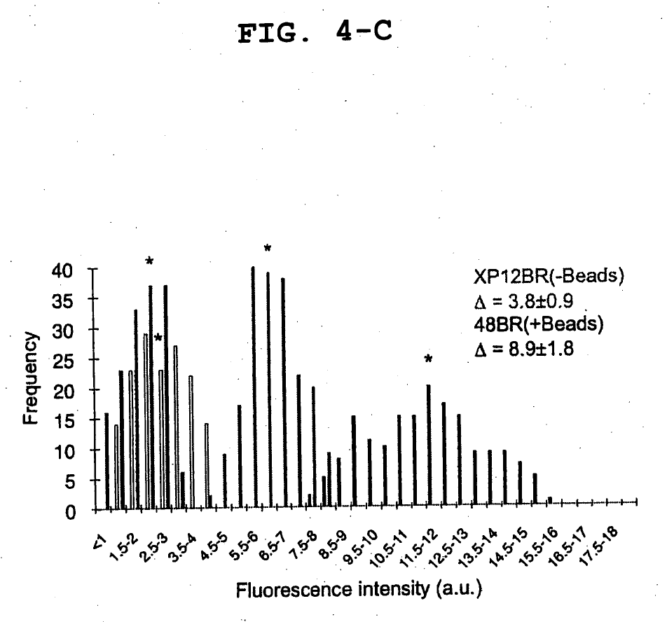

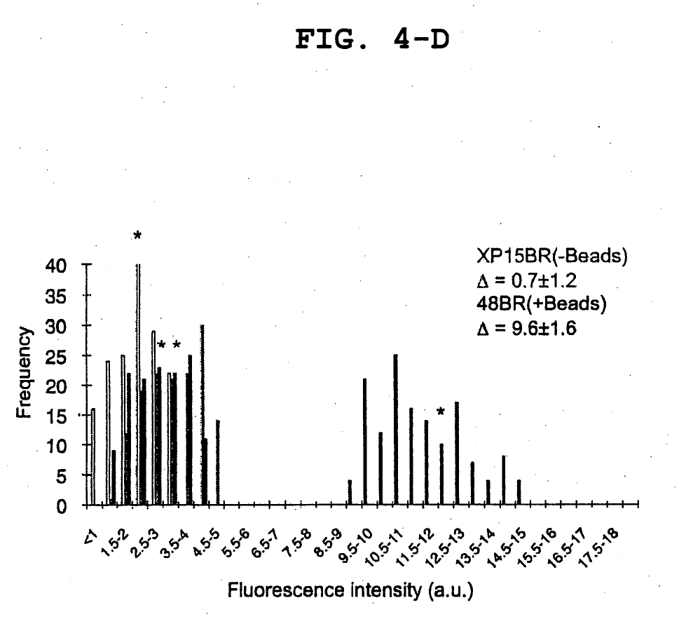

As shown in the upper panel of FIG. 4A and the corresponding histogram (FIG. 4B), the fluorescence intensity in the bead-incorporated 48BR cells were substantially the same as that in the cells without the beads; it was demonstrated that the beads did not interfere with the UDS assay based on EdU incorporation. Both fibroblasts slightly deficient in UDS [XP12BR, FIGS. 4A (middle panel) and 4C] and fibroblasts severely deficient in UDS [XP15BR, FIGS. 4A (lower panel) and 4D] could easily be distinguished from the co-cultured 48BR fibroblasts by the nuclear fluorescence levels thereof. UDS measurements with the internal control and UDS measurements without were almost the same (compare FIGS. 2A, 2C and 2F and FIGS. 4B, 4D and 4C); the inter-experiment or intra-experiment variation of fluorescence intensity among different cover glasses was small in the assay based on EdU incorporation. This feature may be advantageous in applying the EdU assay to experiments where the use of an internal standard is inappropriate (e.g., fluorescence-based high throughput screening using GE's In-Cell-Analyzer or flowcytometry).

### Test Example 5

### Improvement of Sensitivity by Addition of FdU, Extension of Incubation Time, and Acid Extraction

Although the above-described experiments based on EdU incorporation are sufficient for ordinary XP screening and many NER studies, higher sensitivity is required for experiments that require greater accuracy, such as distinguishment between the complete absence of UDS activity and extremely low UDS activity. Hence, attempts were made to increase specific EdU incorporation and reduce nonspecific backgrounds. The incorporation of a thymidine analogue in DNA depends on the concentration thereof related to the thymidine nucleotide endogenously synthesized in a nucleotide pool. Fluorodeoxyuridine (FdU) is an inhibitor of thymidylic acid synthesis, and raises the concentration of a nucleotide derived from externally added thymidine or analogue thereof in the cells. Hence, ultraviolet irradiation was followed by incubation with FdU for a longer time (4 hours). Furthermore, to reduce the background, stringent acid extraction using Bouin's fixative (Sigma) was attempted. The specific procedures used are described below. Normal 48BR cells were cultured on cover glass and irradiated with ultraviolet (20 J/m²), followed by incubation along with 10 μM EdU and 1 μM FdU (Sigma) for 4 hours. The cells were then fixed as shown in Test Example 1. Acid extraction was performed using Bouin's fixative for 30 minutes; subsequently, the cells were thoroughly washed with PBS. Coupling of the fluorescent dye and detection of EdU signals were performed in the same manners as the experiments in Comparative Example 2.

### (Results)

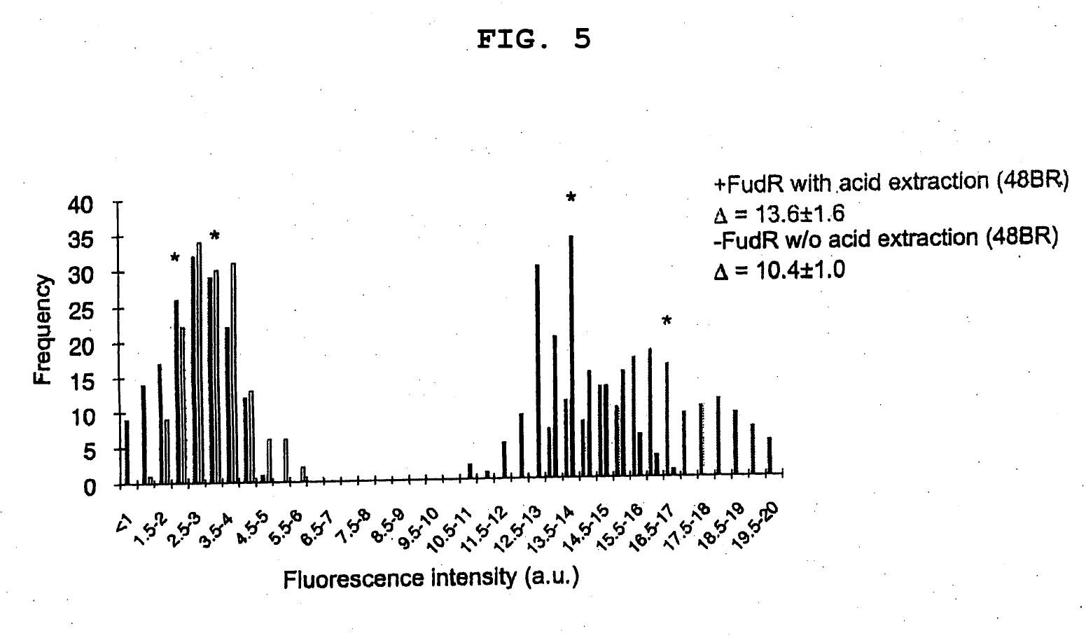

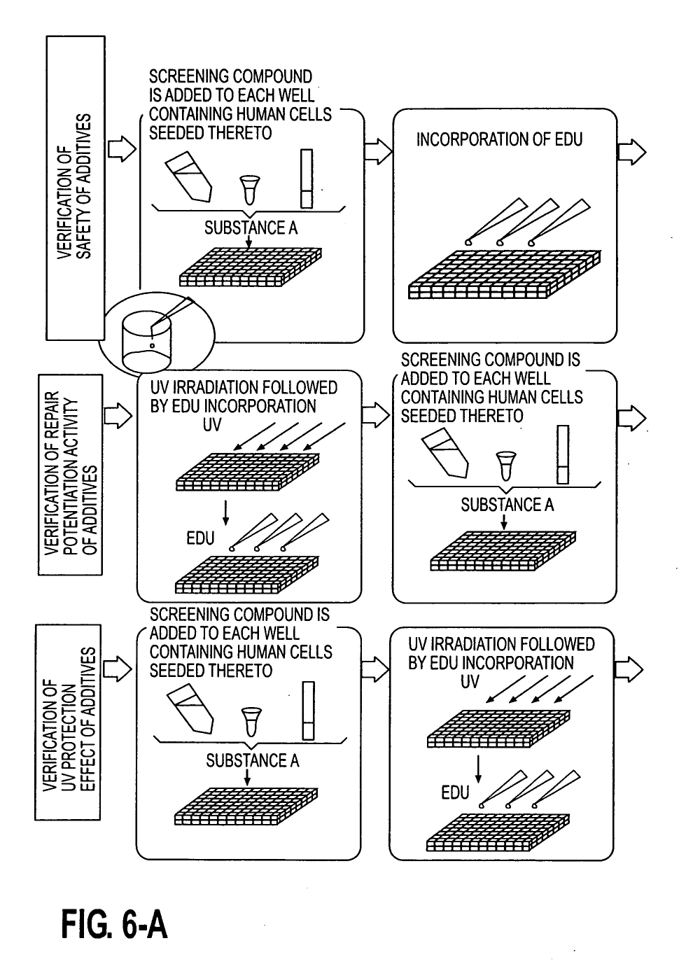

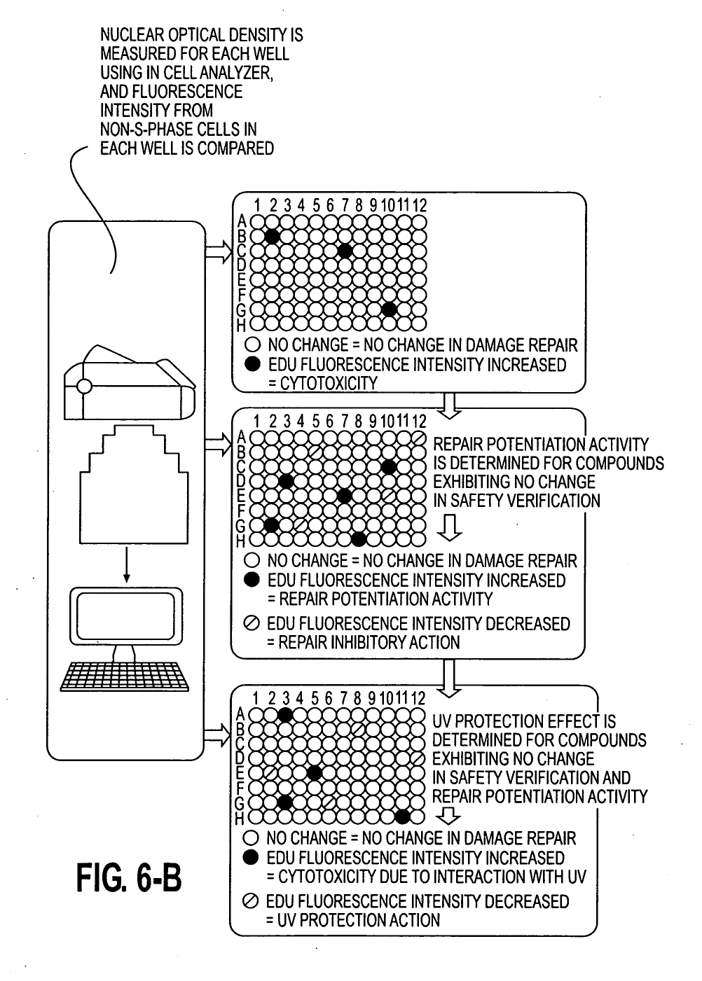

As shown in FIG. 5, both the background (white and red bars and corresponding asterisks thereof compared) and UDS specific EdU incorporation (black and blue bars compared) improved, although the changes were not dramatic. Each bar indicates a frequency of fluorescence level for the indicated class: with ultraviolet irradiation (black, the same as FIG. 2A; blue,+FdU+4 hour incubation+acid extraction) or without ultraviolet irradiation (white, the same as FIG. 2A; red,+FdU+4 hour incubation+acid extraction).

### Example 1

Normal human primary fibroblasts in monolayer culture on a 96-well microtiter plate were washed with PBS and irradiated with ultraviolet light (254 nm) at 20 J/m². Immediately after the ultraviolet irradiation, the cells were cultured, along with a test substance, in a serum-free DMEM supplemented with 10 μM EdU (Invitrogen), for 2 hours. After the cultivation, the cells were washed with PBS, fixed and permiabilized in a PBS containing 2% formaldehyde, 0.5% Triton X-100 and 300 mM sucrose for 20 minutes. After being thoroughly washed with PBS, the cells were blocked with a PBS supplemented with 10% FBS for 30 minutes. The cells were incubated, along with EdU and azide-coupled Alexa Fluor 488 dye, in a TBS supplemented with 4 mM CuSO₄, for 30 minutes. The cells were then washed with a PBS containing 0.05% Tween-20 (PBST) three times. After 100 μl of PBS was added to each well, the cells were fully automatically taken using the In-Cell-Analyser (http://www.gelifesciences.co.jp/catalog/web_catalog.asp?frame5_Value=675) (GE); the images captured were statistically analyzed to calculate the mean nuclear fluorescence intensity.

### Industrial Applicability

Substances capable of repairing damaged DNA selected by the screening method of this application are expected to find applications for cosmetics and/or pharmaceuticals and the like, by, for example, being added to sun block creams and creams having an anti-aging effect for protection against aging due to ultraviolet injury and the like.

This application is based on a patent application No. 2009-172521 filed in Japan, the contents of which are incorporated in full herein by this reference.

1. A screening method for a substance or gene that potentiates the DNA repair capability, a substance or gene that suppresses the induction of DNA damage in DNA-damaged cells, a substance or gene that suppresses the induction of DNA damage, a substance or gene that influences DNA repair, or the presence or absence of a toxicity of a substance with DNA damage as an index, comprising using a reagent set of a terminal alkyne-modified nucleoside derivative and a reporter molecule containing an azide moiety in combination, or a reagent set of an azide-modified nucleoside derivative and a reporter molecule containing a terminal alkyne in combination.

2. The screening method according to claim 1, comprising the following steps (a), (b), (c) and (d):
(a) the step of treating cells with ultraviolet or a mutagen to induce DNA damage,
(b) the step of bringing into contact with each other a test substance, the cells treated in the step (a), and the terminal alkyne-modified nucleoside derivative,
(c) the step of measuring the incorporation of the terminal alkyne-modified nucleoside derivative in the cells after completion of the above-described steps using the reporter molecule containing an azide moiety, and comparing the incorporation with a control group, and
(d) the step of selecting a substance that alters the terminal alkyne-modified nucleoside derivative incorporation rate on the basis of the results of the comparison in the step (c) above.

3. The screening method according to claim 1, comprising the following steps (a), (b′), (c′) and (d′):
(a) the step of treating cells with ultraviolet or a mutagen to induce DNA damage,
(b′) the step of bringing into contact with each other a test substance, the cells treated in the step (a), and the azide-modified nucleoside derivative,
(c′) the step of measuring the incorporation of the azide-modified nucleoside derivative in the cells after completion of the above-described steps using the reporter molecule containing a terminal alkyne, and comparing the incorporation with a control group, and
(d′) the step of selecting a substance that alters the azide-modified nucleoside derivative incorporation rate on the basis of the results of the comparison in the step (c′) above.

4. The screening method according to claim 2, wherein the contact of the cell and test substance takes place before the step (a), the contact of the test substance is completed in the step (a), and the contact of the test substance is skipped in the step (b).

5. The screening method according to claim 3, wherein the contact of the cells and test substance takes place before the step (a), the contact of the test substance is completed in the step (a), and the contact of the test substance is skipped in the step (b′).

6. The screening method according to claim 2, wherein the operation to restrict the expression of a gene in the cells used in the step (a) takes place before the step (a),
the expression of the gene in the cells is restricted in the step (a),
the contact of the test substance is skipped in the step (b), and
a gene that alters the incorporation rate with restricted expression is selected in the step (d).

7. The screening method according to claim 3, wherein the operation to restrict the expression of a gene in the cells used in the step (a) takes place before the step (a),
the expression of the gene in the cells is restricted in the step (a),
the contact of the test substance is skipped in the step (b′), and
a gene that alters the incorporation rate with restricted expression is selected in the step (d′).

8. The screening method according to claim 6, wherein the method is for searching for a gene that potentiates DNA repair capability in DNA-damaged cells, a gene that suppresses the induction of DNA damage, or a gene that influences DNA repair.

9. The screening method according to claim 7, wherein the method is for searching for a gene that potentiates DNA repair capability in DNA-damaged cells, a gene that suppresses the induction of DNA damage, or a gene that influences DNA repair.

10. The screening method according to claim 1, wherein the method is for searching for an active ingredient for a therapeutic agent for a disease accompanied by a DNA repair deficiency or a disease caused by a DNA repair deficiency that has occurred spontaneously in normal humans.

11. The screening method according to claim 10, wherein the disease accompanied by a DNA repair deficiency is xeroderma pigmentosum, Cockayne's syndrome or trichothiodystrophy.

12. The screening method according to claim 1, wherein the method is for searching for an ingredient of a cosmetic or pharmaceutical having an anti-aging effect.

13. The screening method according to claim 12, wherein the cosmetic or pharmaceutical having an anti-aging effect is an ultraviolet-guard cosmetic.

14. The screening method according to claim 1, wherein the method is for screening for the presence or absence of a toxicity of a substance with DNA damage as an index, comprising the following steps (b″), (c″) and (d″):
(b″) the step of bringing into contact with each other a test substance, cells, and the terminal alkyne-modified nucleoside derivative,
(c″) the step of measuring the incorporation of the terminal alkyne-modified nucleoside derivative in the cells contacted with the test substance, using the reporter molecule containing an azide moiety, and comparing this incorporation with the incorporation in control cells not contacted with the test substance, and
(d″) the step of judging a substance that raises the terminal alkyne-modified nucleoside derivative incorporation rate significantly as a substance that exhibits a cytotoxicity on the basis of the results of the comparison in the step (c″) above.

15. The screening method according to claim 1, wherein the method is for screening for the presence or absence of a toxicity of a substance with DNA damage as an index, comprising the following steps (b′″), (c′″) and (d′″):
(b′″) the step of bringing into contact with each other a test substance, cells, and the azide-modified nucleoside derivative,
(c′″) the step of measuring the incorporation of the azide-modified nucleoside derivative in the cells contacted with the test substance, using the reporter molecule containing a terminal alkyne, and comparing this incorporation with the incorporation in control cells not contacted with the test substance, and
(d′″) the step of judging a substance that raises the azide-modified nucleoside derivative incorporation rate significantly as a substance that exhibits a cytotoxicity on the basis of the results of the comparison in the step (c′″) above.

16. A diagnostic method for cells with a DNA repair deficiency, comprising the following steps (A), (B), (C) and (D):
(A) the step of treating cells from a subject with ultraviolet or a mutagen to induce DNA damage,
(B) the step of bringing into contact with each other the cells treated in the step (A) and a terminal alkyne-modified nucleoside derivative,
(C) the step of measuring the incorporation of the terminal alkyne-modified nucleoside derivative in ultraviolet-treated cells, using a reporter molecule containing an azide moiety, and comparing this incorporation with the incorporation in control cells treated to induce DNA damage, and
(D) the step of determining the presence or absence of a DNA repair deficiency in the subject on the basis of the results of the comparison in the step (C) above.

17. A diagnostic method for cells with a DNA repair deficiency, comprising the following steps (A), (B′), (C′) and (D′):
(A) the step of treating cells from a subject with ultraviolet or a mutagen to induce DNA damage,
(B′) the step of bringing into contact with each other the cells treated in the step (A) and an azide-modified nucleoside derivative,
(C′) the step of measuring the incorporation of the azide-modified nucleoside derivative in ultraviolet-treated cells, using a reporter molecule containing a terminal alkyne, and comparing this incorporation with the incorporation in control cells treated to induce DNA damage, and
(D′) the step of determining the presence or absence of a DNA repair deficiency in the subject on the basis of the results of the comparison in the step (C′) above.

18. The diagnostic method according to claim 16, wherein the cells with a DNA repair deficiency are derived from a patient possibly having xeroderma pigmentosum, Cockayne's syndrome or trichothiodystrophy.

19. The diagnostic method according to claim 17, wherein the cells with a DNA repair deficiency are derived from a patient possibly having xeroderma pigmentosum, Cockayne's syndrome or trichothiodystrophy.

20. A diagnostic kit for a disease accompanied by a DNA repair deficiency, comprising a reagent set of a terminal alkyne-modified nucleoside derivative and a reporter molecule containing an azide moiety in combination, or a reagent set of an azide-modified nucleoside derivative and a reporter molecule containing a terminal alkyne in combination.

21. A screening kit for a substance or gene that potentiates repair capability in DNA-damaged cells, a substance or gene that suppresses the induction of DNA damage, a substance or gene that influences DNA repair, or the presence or absence of a toxicity of a substance with DNA damage as an index, comprising a reagent set of a terminal alkyne-modified nucleoside derivative and a reporter molecule containing an azide moiety in combination, or a reagent set of an azide-modified nucleoside derivative and a reporter molecule containing a terminal alkyne in combination.
